(19) United States
(12) Patent Application Publication
(10) Pub. No.: US 2007/0016277 A1
(43) Pub. Date: Jan. 18, 2007

(54) Lip augmentation device

(76) Inventors: Carol Karat, Ra'anana (IL); Israel Tal, Moshav.heebat zion (IL); Daniela Mavor, Tel Aviv (IL); Dalia Jayes, Modiin (IL)

Correspondence Address: KENYON & KENYON LLP, 1500 K STREET N.W., SUITE 700, WASHINGTON, DC, 20005, US

(21) Appl. No.: 11/476,591

(22) Filed: Jun. 29, 2006

Related U.S. Application Data
(60) Provisional application No. 60/695,848, filed on Jul. 5, 2005.

(51) Int. Cl. A61N 1/18

(52) U.S. Cl. 607/134; 128/898; 607/50

(57) ABSTRACT

The present invention is of a method of facilitating lip augmentation, the method comprising treating the lip area with an electricity generating device. The present invention further provides devices and kits for lip augmentation.

This application claims priority to U.S. Provisional Application Ser. No. 60/695,848, filed Jul. 5, 2005, which is incorporated by reference herein in its entirety.

### FIELD AND BACKGROUND OF THE INVENTION

The present invention is of an electricity generating device configured as a device for facilitating lip augmentation.

Lip augmentation is a procedure for enhancing the lips and can include, resizing, such as plumping, reshaping, such as enlargement or lifting of the lips and which can also reduce wrinkles around the mouth. There are several techniques used and described in the art, such as surgical restructuring, injection or application of topical preparations.

Surgical enhancement is an invasive technique and although not complex, includes the risks of any simple surgical procedure. Surgical implants include permanent and reversible implants, such as those made from polyacrylamide or polytetrafluoroethylene. Undesired effects from use of implants can include infection, extrusion, migration of micro implants, palpability and lessened mobility of the treatment areas.

Lip augmentation by injection should preferably be done by a professional in order to achieve the desired effect. Injectables can include bovine and human collagen, fat transferred from another site in the patient's body, fascia lata, hyaluronic acid and hydrophilic polyacrylamide gels. Most injectables result in a temporary effect and treatment needs to be repeated, such as for example with collagen injection, which may last about 3 to 4 months. There are several injectables which are permanent, such as injection of silicone oil and Artecoll's polymethylmethyl acrylate microspheres. The advantage of an injectable is the resulting lip volume, compared to the implants, which give a more subtle defined lip. However, there are problems associated with permanent injectables such as necrosis, granuloma, nidus, severe inflammation, palpability, extrusion and infection.

A further technique is laser lip rejuvenation, which can tighten the natural collagen and elastic tissue beneath the lips.

Lip augmentation can also be achieved using topical preparations. Such topical preparations typically work by slightly inflaming the lip mucosa and can include Capsicum annuum, Capsicum frutescens, cinnamon oil, caffeine, palmitoyl-oligopeptide and 1-arginine. However, the resultant effect is usually very temporary and not very noticeable.

It would therefore be beneficial to have an alternative method of facilitating permanent or temporary lip augmentation with simultaneous elimination of wrinkles around the mouth, which would produce fast results, without the problems or pain associated with the known methods of the art. The present invention provides such a solution.

### BRIEF DESCRIPTION OF THE DRAWINGS

FIG. 1a shows a schematic view of an electricity generating device including a power source for lip augmentation according to one embodiment of the present invention;

FIG. 1b shows a schematic view of a kit for lip augmentation according to an embodiment of the present invention;

FIG. 2 illustrates a schematic representation of an exemplary power source in accordance with an embodiment of the invention;

FIG. 3a shows a schematic view of an electricity generating device for lip augmentation according to an alternative embodiment of the present invention;

FIG. 3b shows a schematic view of a kit including a device of FIG. 3a according to an embodiment of the present invention;

FIG. 4 shows a schematic view of a passive patch for lip augmentation;

FIG. 5a shows a flow chart of a method of lip augmentation using an electricity generating device according to an embodiment of the present invention;

FIG. 5b shows a flow chart of an alternative method of lip augmentation using an electricity generating device according to an embodiment of the present invention;

FIG. 5c shows a flow chart of an alternative method of lip augmentation using an electricity generating device according to an embodiment of the present invention;

FIG. 6 shows one non limiting example of a patch design for lip augmentation according to an embodiment of the present invention; and

FIG. 7 shows one non limiting example of a design for a lip augmentation device according to an embodiment of the present invention.

### DETAILED DESCRIPTION OF EMBODIMENTS

Embodiments of the present invention provide a method of lip enhancement by treating a lip area with an electricity generating device with or without an active composition. The term ‘lip enhancement’ as used herein includes, but is not limited to lip resizing, such as plumping, lip reshaping, such as enlargement or improving symmetry or lifting of the lips and which can also include reduction of wrinkles of the lip area including around the mouth. The term ‘lip area’ as used herein includes any suitable lip area such as the upper lip, lower lip, internal mouth lip region (inner lip), external mouth lip region (outer lip), lip contour, skin surrounding the lip region and any combination thereof. Electricity generating devices configured to deliver a current to a body region and/or deliver an active composition to a body region can work using various mechanisms, which can include for example iontophoresis, electroosmosis and electroporation to deliver electrical current to a body region and promote delivery of an active agent. Iontophoresis is an effective and painless method of delivering cosmetic and pharmaceutical active agents to a localized tissue area by applying electrical current to a formulation of the medication.

Typically, an electricity generating device can include at least 2 electrodes, wherein there is a potential difference between the 2 electrodes and wherein the electrodes are electrically and/or ionically coupled together. In some embodiments, an electricity generating device can further include a power source. In some embodiments, electrical stimulation of the lip area can facilitate increased blood flow and vasodilation for a lip augmentation effect. In an embodiment, wherein the electricity generating device includes an active composition, the method of the present invention can facilitate promoting delivery of the active composition to the lip area for a lip augmenting effect.

As used herein the term ‘electricity generating device’ includes, but is not limited to any suitable electricity generating device of any size or bulk, such as a powered dermal patch, dermal device, patch device, a galvanic stimulation device or any delivery device, or electrical stimulating device, which is powered and can facilitate a change in a skin or body condition, by any suitable mechanism. The term includes, but is not limited to a topical delivery iontophoretic device, topical delivery ultrasonic device, topical delivery RF device, topical delivery micro-needle device, topical physical therapy device, such as, but not limited to TENS, MIMS, electric wound healing device, skin stimulator, neural stimulator, muscle stimulator, oral care stimulator diffuse surface treatment device and a combination thereof.

Embodiments of the present invention also provide a method of lip enhancement using the combination of the effect of the electricity generating device and heat.

Embodiments of the present invention further provide a method of lip enhancement using a passive patch. A passive patch can facilitate occlusion of the lip area to be treated and can be advantageous when compared to topical application and treatment.

Embodiments of the present invention also provide a device such as an electricity generating device or passive patch configured for lip augmentation. Moreover, the present invention can be a fully integrated electricity generating device for lip augmentation, featuring at least one delivery electrode and a counter electrode, wherein there is a potential difference between the delivery and counter electrodes, a conductive substance layer, such as, but not limited to a hydrogel, and an active substance, wherein the device components are disposed on a base layer substrate. In some embodiments the electricity generating device includes a power source. Embodiments of the present invention further provide a kit including a device such as described above, wherein the active substance and/or hydrogel are not attached to the device before use.

The present invention provides a method of lip augmentation, which is facile to use without the need for professional administration, facilitating an immediate effect on the lip area, which can be temporary or permanent.

The principles and operation of a device and method according to the present invention may be better understood with reference to the figures. The figures show non-limiting embodiments of the present invention.

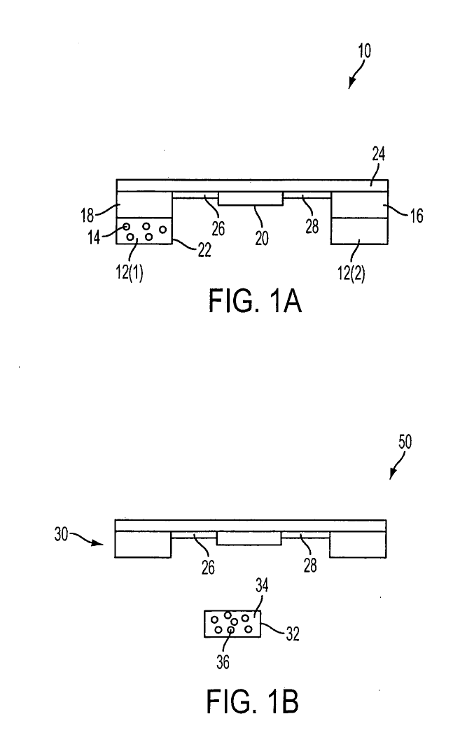

FIG. 1a shows a schematic view of an exemplary electricity generating device for lip augmentation according to one embodiment of the present invention. As stated above electricity generating device can be of any size or thickness or type of electricity generating device. FIG. 1a illustrates one example, wherein electricity generating device is a patch. The device 10 can be fully integrated in the sense that the conductive substance/layer 12 and lip augmentation active substance 14 is incorporated into the device. In this embodiment, device can be a patch 10, which may comprise at least one first electrode 16, identified as “cathode,” at least one second electrode 18, identified as “anode”, electrochemical cell 20 as the power source of patch 10, at least one holding means 22 for accommodating a conductive substance 12 and at least one active substance 14 and at least one conductive substance 12 (1), 12 (2). The term ‘holding means’ as used here includes any type of means, including a retainer and a separator for accommodating a conductive substance/carrier and/or an active substance. The term can include a conductive substance which can accommodate an active substance. Optionally, patch 10 may include a plurality of cathodes 16, a plurality of anodes 18 and a plurality of power sources 20. In some embodiments, patch 10 may comprise conductive substance/s 12(1), 12(2) to provide an interfacing layer between patch 10 and a lip area of a subject. Optionally, patch 10 may not include a holding means 22. In such an embodiment, active substance can be disposed directly on delivery electrode.

As shown in FIG. 1a, electrodes 16, 18, holding means 22, conductive substance 12(1), 12(2), active substance 14 and electrochemical cell 18 may be supported on a base layer substrate 24. Electrode 16 may be disposed in any suitable way on substrate 22 in spaced relation to electrochemical cell 20 and electrode 18 to define a gap between the two electrodes 16, 18. Optionally, the same conductive layer 12 can be disposed on both anode 18 and cathode 16 or conductive layers 12(1) and 12(2) can be different conductive substance layers. In some embodiments, a conductive substance, such as a hydrogel 12 is disposed in/on the holding means 22 on the main delivery electrode and any suitable conductive substance, which can facilitate providing an adhesive conductive interface is disposed on the counter electrode. In some embodiments, hydrogel 12 is an aqueous hydrogel.

In some embodiments, patch 10, including patch components, is thin and flexible, to suit the contour of the lip area of a subject. In some embodiments, patch 10 is electrically powered. Patch may optionally be any size, color and shape suitable for application to a lip area. The device 10 can be configured for use on the outer lip (outside the mouth), on the inner lip (inside the mouth) or a combination of inside and outside the mouth. The device 10 can be configured for use on only one of the lip regions or on both lip regions. In some embodiments the device may be configured for use on an upper or lower lip area which may include hair.

Device 10 can be of any thickness. The patch 10 shown in FIG. 1a can be in some embodiments up to about 10 mm to ensure flexibility, but may be thicker, depending on the application. The thickness of the patch may also be dependent upon the type of material used and the flexibility of that material. Patch 10 is in some embodiments disposable, but may be reusable. Patch 10 is stable to a wide range of temperatures and humidity. In some embodiments patch can be biocompatible.

Any power source 20, including at least one electrochemical cell, or galvanic cell or electrolytic cell of any suitable size or shape, which provides an electrical potential of between about 0.2 Volt and about 100 Volt can be used according to the present invention. In some embodiments, power source 20 is an electrical battery, providing an electrical potential of between about 0.5 Volt and 12 Volts. In some embodiments power source 20, can supply a voltage of 1.5 V or 3V. In some embodiments, power source 20 is thin and flexible.

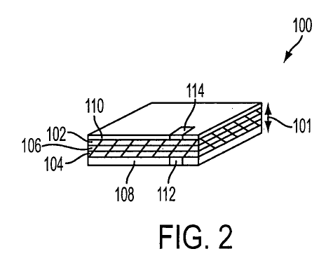

FIG. 2 illustrates a schematic representation of an exemplary power source 100 in accordance with an embodiment of the invention, which is in no way limiting. In some embodiments, power source 100 is thin and flexible. The term “power source” as used herein includes, but is not limited to, any suitable cell in which chemical energy is converted to electric energy by a spontaneous electron transfer reaction. The term includes cells with non-spontaneous reactions, galvanic cells, electrolytic cells, and/or a combination thereof. In the embodiment of FIG. 2, the power source is depicted as an electrochemical cell. The thickness 101 of the electrochemical cell 100 may be up to about 4 mm, in some embodiments up to about 2 mm and in some embodiments up to about 1 mm.

In one embodiment, electrochemical cell 100 includes a positive pole layer 102, a negative pole layer 104, and an electrolyte layer 106 interposed therebetween. In some embodiments, electrochemical cell 100 includes one or more additional conductive layers 108 and 110 to improve the conductivity of pole layers 102 and 104. Suitable conductive layers 108 and 110 are in some embodiments made from any suitable conductive material, such as carbon, graphite, silver, platinum or gold or combinations thereof. In some embodiments conductive layers (current collectors) 108 and 110 are graphite or carbon based layers, which can be printed or applied in any suitable way to cell 100. Examples of graphite and carbon based layers include graphite or carbon webs, sheets, inks and cloth. In some embodiments, electrochemical cell includes negative terminals 112 and positive terminals 114, which are in contact with the corresponding pole layer 104 and 102 or with the corresponding conductive layer 108 and 110 or both. Terminals are made of any suitable material such as, but not limited to, graphite or metal and are in some embodiments applied to cell 100 by a suitable printing technology. Terminals may be located in any desired location of cell 100 and may acquire any suitable shape and size, depending on the specific application. Optionally, terminals may protrude from the surface of cell 100.

By way of example, a suitable electrochemical cell 100 is described in U.S. Pat. Nos. 5,652,043, 5,897,522, and 5,811,204, each of which are incorporated herein by reference in their entireties. Briefly, the electrochemical cell described in the above-identified U.S. patents is an open liquid state, electrochemical cell, which can be used as a primary or rechargeable power source for various miniaturized and portable electrically powered devices of compact design. In one embodiment, an electrochemical cell 100 may comprise a first layer of insoluble negative pole 104, a second layer of insoluble positive pole 102, and a third layer of aqueous electrolyte 106 disposed between the first 104 and second 106 layers and may include (a) a deliquescent material (not shown) for keeping the open cell wet at all times; (b) an electroactive soluble material (not shown) for obtaining required ionic conductivity; and, (c) a polymer (not shown) for obtaining a required viscosity for adhering the first and second layers to the third layer.

Yet, in another preferred embodiment, an electrochemical cell may comprise a plurality of self-contained, serially connected galvanic power sources, as described for example in U.S. Pat. No. 6,421,561, which is incorporated herein by reference in its entirety.

Preferred configurations for electrochemical cells according to the present invention involve those combinations, which are devoid of poisonous compounds.

In some embodiments, the power source is applied using a suitable printing technique.

Referring back to FIG. 1a, power source 20 in patch 10 may be a single electrochemical cell. However, power source 20 need not be limited to one cell, but may include a plurality of connected electrochemical cells, a plurality of batteries, and/or electronics configured to increase, control, and change phase of the supplied electric current and wherein the power source is thin and flexible. Electrochemical cell 20 in patch 10 in some embodiments provides electrical potential (voltage) to the desired lip area of the subject. In some embodiments, the electrical potential may be adjusted to satisfy at least one of the following three criteria.

First, the patch voltage may be adjusted to enable an iontophoretic delivery or other mechanism of delivery of active substance into the lip area. Second, the patch voltage may be adjusted to minimize the penetration of the active substance through the lip, and to maximize the amount into the desired lip area. Third, the patch voltage may be adjusted to minimize lip area irritation, which may result from excessive electric current, passing into and through the lip or optimize patch voltage in order to cause lip irritation for lip augmentation effect.

The power source 20 may optionally be located in any suitable position on the patch 10.

Current provided by the electricity generating device 10 configured for drug delivery, such as a patch may be any suitable form of current, including DC, AC, pulse or other phase form.

Cathode and anode electrodes 16 and 18 are in some embodiments composed of a conductive material. Any suitable conductive material can be used, such as, but not limited to silver, silver/silver chloride, zinc, manganese dioxide, graphite, carbon, copper, aluminium, platinum, stainless steel, tin oxide, silver oxide, gold and titanium, hydrophobic polymer matrix containing a conductive filler such as metal powder/flakes, powdered graphite, carbon fibers or other known electrically conductive filler material and combinations thereof. In some embodiments, at least one electrode is a main delivery electrode and at least one electrode is a counter electrode. Optionally, the main electrode can be the cathode or anode or both the cathode and the anode. Defining which electrode is the main electrode is dependent on the charge of the ions contained in the conductive/active agent formulation 12(2), 14 disposed on the main delivery electrode 18. In one embodiment, anode 18 is the main electrode and cathode 16 is the counter electrode. In an alternative embodiment, cathode 16 is the main delivery electrode and anode 18 is the counter electrode. In an embodiment, wherein device 10 includes at least one holding means disposed on the anode and at least one holding means disposed on the cathode and each holding means includes an active formulation, with opposite charged ions, both anode 18 and cathode 16 can be delivery electrodes.

Electrodes 16 and 18 may optionally be provided in any suitable form, such as, but not limited to as thin sheets, linked to the power source, or printed onto a substrate in spaced relation to each other to define a gap therebetween. Optionally, the electrode area can be continuous, or formed in any shape or configuration. Optionally, cathode 16 and anode 18 may not have the same shape and/or same area. Optionally, cathode and anode may be in any suitable conformation in relation to each other including but not limited to a coplanar and cofacial arrangement. Optionally, patch can include a plurality of anodes and a plurality of cathodes.

In some embodiments, cathode 16 and anode 18 are connected to battery 20 by any suitable connection means, 26, 28, such as electrical conduction means/media. Examples of connection means 26, 28, include, but are not limited to wiring, conductive ink, conductive adhesive tape, printed connection means, soldered connection means, connection means attached by UV, glued connection means and a combination thereof.

Substrate base layer/frame 24 is optionally any suitable material, which can accommodate the patch components. Suitable materials include, but are not limited to woven material, non-woven material, polymers, conducting material, non-conducting material, paper, cardboard, plastic, synthetic materials, natural materials, fabric, metals, wood, glass, Perspex, or a combination thereof. In some embodiments, substrate material is a non-conductive material. In some embodiments, substrate is made from polyester. In some embodiments, the shape of the substrate base layer can be configured according to the shape of the electrodes 16, 18. In some embodiments, the shape of the substrate base layer 24 is configured to be ergonomic with the shape of the lip area region where the patch 10 is to be used.

Optionally, substrate base layer 24 may readily facilitate attachment of the device 10 to a desired lip area. Attachment mechanisms may include but are not limited to conductive adhesive, adhesive strip, release liners, suction cups and/or any combinations thereof. In the embodiment of FIG. 1a, patch 10 is configured to attach to the lip area by conductive layers 12(1), 12(2). In some embodiments, the patch may be attached to the lip area by, for example, the frame of the substrate and/or other attachment mechanisms.

Conductive substance layers 12(1) and 12(2) may optionally be any suitable conductive composition/fluid, such as an aqueous gel, hydrogel or a conductive adhesive. It is appreciated that non-aqueous conductive fluids may also be employed. Optionally, the hydrogel or other conductive substance can be anhydrous or in a dehydrated state. In such an embodiment, water can be added prior to use. In some embodiments, conductive substance 12 (1) readily facilitates providing at least one of or a combination of the following functions: a conductive interfacing layer between the lip area and electrode 18; a conductive adhesive facilitating attachment of the patch to the skin.

In some embodiments conductive substance 12 can include at least one additional formulation, which can optionally include at least one active composition for lip enhancement 14. Any lip enhancement formulation in any suitable form as known in the art including liposome and nanosome formulations can be used with the device of the present invention. Non limiting examples include Capsicum annuum, Capsicum frutescens, cinnamon oil, caffeine, palmitoyl-oligopeptide, I-arginine bovine and human collagen, fat transferred from another site in the patient's body, fascia lata, hyaluronic acid and hydrophilic polyacrylamide gels, polytetrafluoroethylene, silicone oil and Artecoll's polymethylmethyl acrylate microspheres. Other non limiting examples of formulations which can be included in the present invention can be found on www.lipaugmentation.com. The device can also include active compositions, such as but not limited to bleaching agents, hair removal agents, lip coloring agent, moisturizing agents, anti-wrinkle agents, anti-acne agents or combinations thereof. Device of the present invention can promote delivery of the active composition to produce a lip augmenting effect. The electrical stimulation effect of device 10 and the effect of the lip augmenting formulation can result in a synergistic lip augmenting effect. In some embodiments, wherein device includes an additional or alternative active agent, which for example is a bleaching agent, the device can further facilitate bleaching of the lip and/or upper lip skin or hair. In some embodiments wherein device includes an active agent which is an anti-wrinkle agent, the device can promote an anti wrinkle effect. In some embodiments, wherein device includes an active agent which is an anti-wrinkle agent, the device can promote an anti wrinkle effect in addition to the anti wrinkle effect facilitated by the lip plumping active agent. In some embodiments, wherein device includes an active agent which is a lip coloring agent, the device can facilitate lip augmentation and lip coloring.

In some embodiments, device 10 can include a heating means (not shown in figures). Heating means can be any suitable heating means, such as but not limited to an exothermic heating component whereby device can facilitate heating the lip area and enhancing the lip augmentation effect. Without wishing to be limited by a single mechanism, heating may produce a lip augmentation effect by facilitating vasodilation. In addition heating may improve the permeability of the lip area to an active lip augmenting composition.

In some embodiments, device 10 can include a holding means 22 for accommodating a formulation, which can include a conductive gel 12 and an active substance 14. Optionally, holding means can include a retainer/substrate made of a porous non-conductive material, such as, but not limited to a sponge, pad, paper, non-woven polypropylene etc, that serves to retain the conductive fluid therein. One non-limiting example of a holding means 22 is a hydrogel, which can accommodate an active formulation. Optionally, when device 10 is not a thin and flexible patch, holding means can be a chamber for accommodating the active formulation.

In one embodiment, holding means 22 can only include conductive formulation 12 and active substance 14 can be applied as a separate layer optionally on the holding means or directly on the lip area region to be treated.

As noted, the embodiment depicted in FIG. 1a is a fully integrated patch device configured to facilitate promoting delivery of an active substance. The present invention may also be practiced with a device 30 that does not have a holding means with conductive substance and active agent incorporated into it, but which instead is part of a kit 50, such as shown in FIG. 1b. Device can be any suitable electricity generating device. For illustrating purposes, FIG. 1b shows one exemplary embodiment, wherein device 30 is a patch. FIG. 1b shows a patch device 30, which is similar to the embodiment of the fully integrated patch device depicted in FIG. 1a and described hereinabove. However, in this alternative embodiment, the holding means 32 and conductive substance 34 including active formulation 36 disposed therein of the patch is optionally not integrally formed with the patch 30. Optionally, when the patch device 30 is part of a kit 50, holding means 32 can be a separate component from the patch device 30. Optionally, separate holding component 32 can be attached to patch 30 just before use, such as for example when separate holding component 32 is a chamber. Alternatively, separate holding component 32 can be applied onto the lip area, such as for example when separate holding component 32 is a sponge or other type of material absorbing device. Alternatively, conductive substance 34 and active agent 36 may be applied directly onto lip area or onto electrode, without use of a separate holding component.

Certain features of the patch of the present invention 10 are the same regardless of whether the patch 10 is a fully integrated patch device or a patch 30 that is part of a kit, such as, but not limited to the substrate base layer/frame, cathode, anode, power source, battery and electrode connections, liner and battery cover, which have been described hereinabove.

In an alternative embodiment (not shown in FIGS. 1a and 1b), electricity generating device is used without an active substance. Device 10 without an active substance can facilitate lip augmentation by electrical stimulation causing irritation of the lip area and/or by increasing blood flow in the lip area and/or by increasing water delivery to the lip area.

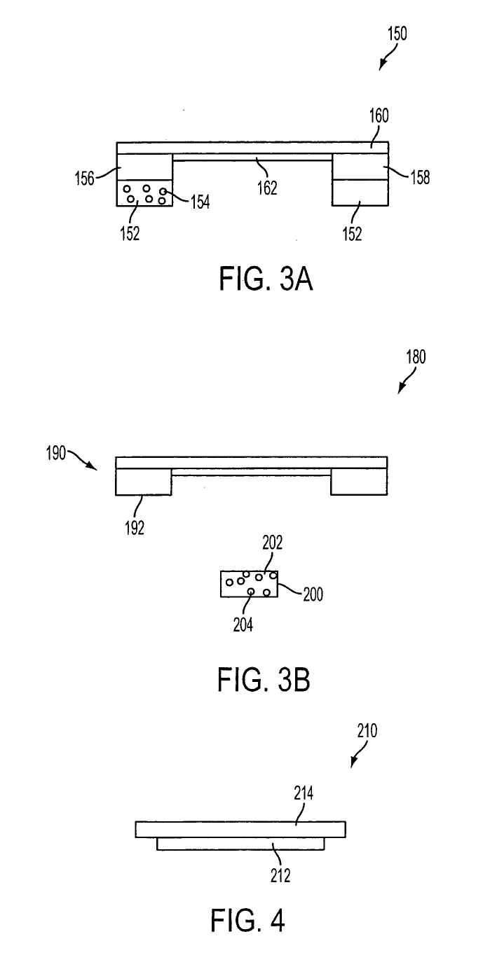

FIG. 3a shows a schematic view of an exemplary electricity generating device for lip augmentation according to an embodiment of the present invention. As stated above electricity generating device can be any size or thickness or type of electricity generating device. FIG. 3a illustrates one example, wherein electricity generating device is a patch. The device 150 can be fully integrated in the sense that the conductive substance/layer 152 and lip augmentation active substance 154 is incorporated into the device. In this embodiment, device 150 can include at least one delivery electrode 156, at least one counter electrode 158, at least one conductive substance 152, and at least one active substance 154 disposed on a base layer substrate 160. In some embodiments at least one delivery electrode 156 and at least one counter electrode 158 comprise electroactive materials such that there is a potential difference between the at least one delivery electrode 156 and the at least one counter electrode 154. The at least one delivery electrode may be electrically coupled 162 or ionically coupled or electrically and ionically coupled to the at least one counter electrode. Electrical coupling 162 may be facilitated by any suitable electrical connection means such as, but are not limited to wiring, conductive ink, conductive adhesive tape, printed connection means, soldered connection means, connection means attached by UV, glued connection means and a combination thereof. Ionic coupling may be facilitated by any suitable ionic medium, such as a hydrogel or other conductive substance layer.

The potential difference facilitates current flow, such that when the device is applied to a lip area, the current can promote delivery of a lip augmenting formulation to facilitate lip augmentation. The description of the patch components are as described in FIGS. 1a and 1b.

The present invention may also be practiced with a kit 180 as shown in FIG. 3b, which includes a device 190 that does not include a conductive substance 202 and/or active agent 204 incorporated into it, but which instead is included in a non-integral, separate holding component 200. Optionally, separate holding component 200 can be attached to patch 190 just before use, such as for example when separate holding component 200 is a chamber. Alternatively, separate holding component 200 can be applied onto the lip area, such as for example when separate holding component 200 is a sponge or other type of material absorbing device. Alternatively, conductive substance 202 and active agent 204 may be applied directly onto lip area or onto electrode 192, without use of a separate holding component 200.

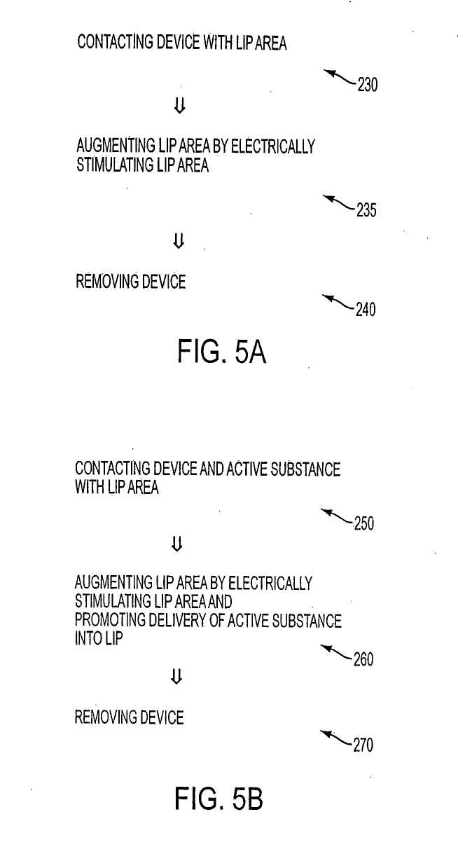

FIG. 4 shows an alternative embodiment of the device of the present invention for facilitating lip augmentation. In the embodiment shown in FIG. 4, device 210 is of a passive patch for facilitating lip augmentation. Device 210 can include an active composition for lip augmentation 212 disposed on a base layer 214. In some embodiments, device 210 can include a rate controlling membrane which is contacted with the lip area and which controls the release of the active composition for lip augmentation 212. In some embodiments, device 210 includes a matrix, such as but not limited to a polymer or polymer adhesive or cotton pad in which the active composition is disposed and which is contacted with the lip area. In some embodiments base layer 214 includes attachment means (not shown in figure). Device 210 is configured to provide occlusion of the lip area and to prevent evaporation of the active composition 212. As such device 210 can result in an improved effect compared to application of a topical preparation.

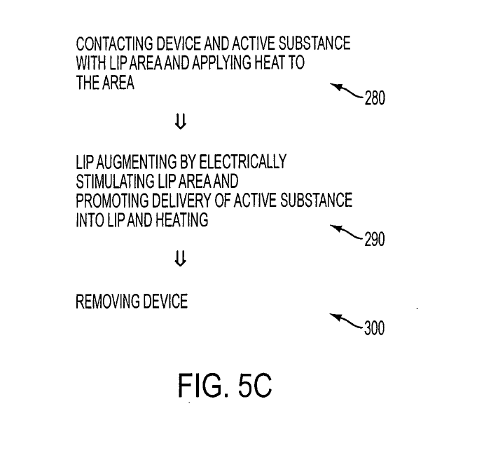

FIG. 5a shows a flow chart of an exemplary method of use of the device of the present invention for lip augmentation. The flowchart applies to a method of use of a fully integrated patch device for electrical stimulation to facilitate lip augmentation. An electrically powered device, such as any of the devices including a patch as herein described, may be provided. In some embodiments, the electrically powered device includes at least one first electrode, and at least one second electrode and at least one power source, supported on a base member in spaced relation to each other to define a gap therebetween and a conductive formulation. The patch may be configured to facilitate providing an electrical current.

In some embodiments, electrically powered device includes at least one first electrode, and at least one second electrode, wherein there is a potential difference between the at least first and at least second electrode, supported on a base member in spaced relation to each other to define a gap therebetween and a conductive formulation disposed on at least one of the electrodes.

The subject may contact a lip area to be treated with the electrically powered device 230.

In some embodiments, the contact of the device with the lip area facilitates current flow and lip augmentation can be facilitated by electrical stimulation 235.

The device is removed from the lip area at the end of treatment time 240. Time of treatment can vary. The device is in some embodiments removed from contact with the lip area after a time period, which can optionally be predetermined or is determined according to the time it takes for the electrode to be depleted, or until sufficient effect or no more improvement can be seen.

In some embodiments a pretreatment can be applied prior to use of the device. Non-limiting examples of pretreatments include applying a cleanser, applying a moisturizing composition, applying a formulation comprising a pharmaceutically active ingredient, applying a formulation comprising a cosmetically active ingredient or a combination thereof.

In some embodiments a post treatment can be applied to the body area after application of the device. Non-limiting examples of post treatments include applying an occlusion formulation, applying a cleanser, applying a moisturizing composition, applying a formulation comprising a pharmaceutically active ingredient, applying a formulation comprising a cosmetically active ingredient or a combination thereof.

FIG. 5b is a flow chart of an exemplary method of use of a device for augmentation of lips according to embodiments of the present invention.

The flowchart applies to a method of use of a fully integrated patch device for promoting delivery of an active substance. An electrically powered device, such as any of the devices including a patch as herein described may be provided. In some embodiments, the electrically powered device includes at least one first electrode, and at least one second electrode and at least one power source, supported on a base member in spaced relation to each other to define a gap therebetween and a holding means for accommodating a conductive formulation and at least one active composition. Optionally, the conductive formulation and active composition can be disposed without a holding means. The patch may be configured to facilitate providing an electrical current and delivering an active lip augmenting agent.

In some embodiments, electrically powered device includes at least one first electrode, and at least one second electrode, wherein there is a potential difference between the at least first and at least second electrode, supported on a base member in spaced relation to each other to define a gap therebetween and a holding means for accommodating a conductive formulation and at least one active composition. Optionally, the conductive formulation and active composition can be disposed without a holding means.

The subject may contact a lip area to be treated with the electrically powered device 250. In some embodiments, electrically powered device is a thin and flexible device, which conforms to the contours of the lip area and which includes attachment means, for ready attachment to the lip area to be treated.

In some embodiments, the contact of the device with the lip area facilitates current flow and promotes delivery of lip augmenting active agent 260. Lip augmentation can be facilitated by electrical stimulation and by active agent effect.

The device is removed from the lip area at the end of treatment time 270. Time of treatment can vary. The device is in some embodiments removed from contact with the lip area after a time period, which can optionally be predetermined or is determined according to the desired dosage, the time it takes for the electrode to be depleted, or until sufficient effect or no more improvement can be seen.

In some embodiments a pretreatment can be applied prior to use of the device. Non-limiting examples of pretreatments include applying a cleanser, applying a moisturizing composition, applying a formulation comprising a pharmaceutically active ingredient, applying a formulation comprising a cosmetically active ingredient or a combination thereof.

In some embodiments a post treatment can be applied to the body area after application of the device. Non-limiting examples of post treatments include applying an occlusion formulation, applying a cleanser, applying a moisturizing composition, applying a formulation comprising a pharmaceutically active ingredient, applying a formulation comprising a cosmetically active ingredient or a combination thereof.

In an embodiment wherein a kit is used the active composition and/or conductive formulation can be attached to the device before use, or can be applied directly to the lip area or can be applied to the delivery electrode of the device.

FIG. 5c shows a flow chart of an alternative method of use of the device of the present invention for lip augmentation. The flowchart applies to a method of use of a fully integrated patch device with a heating means for promoting delivery of an active substance for lip augmentation. An electrically powered device, such as any of the devices including a patch as herein described may be provided. In some embodiments, the electrically powered device includes at least one first electrode, and at least one second electrode and at least one power source, supported on a base member in spaced relation to each other to define a gap therebetween and a holding means for accommodating a conductive formulation and at least one active composition. The device can include any suitable heating means. Optionally, the conductive formulation and active composition can be disposed without a holding means. The patch may be configured to facilitate providing an electrical current and delivering an active lip augmenting agent. Optionally, the device does not include at least one power source. In such an embodiment, the at least first and at least second electrodes are chosen such that there is a potential difference between the two electrodes.

The subject may contact a lip area to be treated with the electrically powered device 280. In some embodiments, electrically powered device is a thin and flexible device, which conforms to the contours of the lip area and which includes attachment means, for ready attachment to the lip area to be treated.

In some embodiments, the contact of the device with the lip area facilitates current flow and promotes delivery of lip augmenting active agent 290. Lip augmentation can be facilitated by electrical stimulation and by active agent effect. In some embodiments, heating means is configured to facilitate heating of the lip area to produce a lip plumping effect. The resulting lip augmentation may be a combination of the electrical stimulation, active formulation and heating effects or may be a synergistic effect.

The device is removed from the lip area at the end of treatment time 300. Time of treatment can vary. The device is in some embodiments removed from contact with the lip area after a time period, which can optionally be predetermined or is determined according to the desired dosage, the time it takes for the electrode to be depleted, or until sufficient effect or no more improvement can be seen.

In some embodiments pretreatments and post treatments may be applied to the lip area.

The treatment using any of the exemplary methods described in FIGS. 5A, 5B and 5C or any other suitable method of lip augmentation using a device as herein described, can optionally be a one-time treatment or can be repeated in suitable time intervals any suitable number of times. Use of the present invention can facilitate temporary lip augmentation or permanent lip augmentation. Duration of effect can be affected by time and frequency of application, dose of active agent, type and amount of current used, user's lip parameters and severity of condition. In one embodiment, the device is configured for home use. In other embodiments, the device can be applied in a supervised environment.

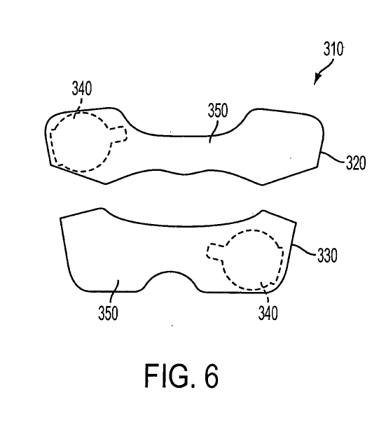

FIG. 6 shows one non limiting example of a patch design 310 for lip augmentation according to the present invention. FIG. 6 is a split treatment patch, wherein one patch 320 is for treatment of the upper lip and a second patch 330 is for treatment of the lower lip. Each of patches 320 and 330 comprise at least one delivery electrode (not shown in figure) and at least one counter electrode (not shown in figure) and a power source 340 disposed on a substrate base layer 350. In an alternative design not shown, both 320 and 330 can be combined in a single patch powered by one power source.

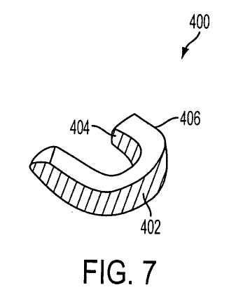

FIG. 7 shows an alternative non-limiting design of an electricity generating device for lip augmentation 400. Device 400 is configured in a similar design to a gum shield, such that it includes a plurality of sides, a front side 402, a back side 404 and an underside (not seen in figure). Between the top of front side 402 and back side 404 is a cavity 406 for accommodating a lip (not shown in figure). The device is attached to the lip, such that the lip is accommodated in cavity 406. Device includes electricity generating components as described herein above for FIGS. 1a, 1b, 2, 3a, 3b and 4. A plurality of devices 400 can be used such that one is applied to the top lip area and a second device is applied to the bottom lip area. In an alternative embodiment (not shown in FIG. 7), a single device may include a top lip insert and a bottom lip insert.

It is to be understood that the invention is not limited in its application to the details of construction and the arrangement of the components set forth in the description. The invention includes other embodiments and can be practiced or implemented in various ways. Also it is to be understood that the phraseology and terminology employed herein is for the purpose of description only and should not be regarded as limiting.

What is claimed is:

1. A method of facilitating lip augmentation, the method comprising treating the lip area with an electricity generating device.

2. The method of claim 1, wherein the lip area is at least one of the outer lip, the inner lip, the top lip, the lower lip, the upper lip, skin surrounding the lip and mouth and a combination thereof.

3. The method of claim 1, wherein the electricity generating device is selected from the group consisting of a powered dermal patch, a dermal device, a patch device, a galvanic stimulation device, a delivery device, an electrical stimulating device, and a combination thereof.

4. The method of claim 1, wherein the electricity generating device facilitates electrical stimulation of the lip area.

5. The method of claim 1, further comprising treating the lip area with at least one active composition and wherein the electricity generating device promotes delivery of the active composition onto and/or into the lip area.

6. The method of claim 5, wherein the at least one active composition is selected from the group consisting of a lip augmenting composition, a bleaching agent, a hair removal agent, a moisturizing agent, an anti-wrinkle agent, an anaesthetic, a lip coloring agent and a combination thereof.

7. The method of claim 1, further comprising treating the lip area with heat.

8. The method of claim 1, wherein treating the lip area comprises applying the electricity generating device to contact the lip area and removing the electricity generating device at the end of the treatment time.

9. The method of claim 1, wherein the electricity generating device comprises at least one first electrode, at least one second electrode, and at least one conductive fluid coupled to at least one of the at least one first electrode and the at least one second electrode; wherein there is a potential difference between the at least one first and second electrodes and wherein the at least one first and second electrodes are in electrical communication and wherein the conductive fluid is in communication with the lip area.

10. The method of claim 9, wherein the electricity generating device further comprises a power source connected to the first and second electrodes.

11. The method of claim 9, wherein at least one of the electrodes is disposed in a region inside the mouth.

12. The method of claim 1, wherein lip augmentation is at least one of enhancing the lips, resizing, plumping, reshaping, enlargement of the lips, improving symmetry, lifting of the lips, reducing lip area wrinkles and reducing wrinkles around the mouth.

13. A method of facilitating lip augmentation, the method comprising treating the lip area with a patch comprising a lip augmenting composition.

14. An electricity generating device for lip augmentation.

15. The electricity generating device of claim 14 comprising at least one delivery electrode, at least one counter electrode, and at least one conductive carrier; wherein there is a potential difference between the at least one delivery and counter electrodes and wherein the at least one delivery electrode and the at least one counter electrode are in electrical communication and wherein the conductive carrier is coupled to at least one of the at least one delivery electrode and the at least one counter electrode and is in communication with the lip area.

16. The electricity generating device of claim 14 comprising at least one active composition.

17. The electricity generating device of claim 16, wherein the at least one active composition is selected from the group consisting of a lip augmentation agent, a bleaching agent, a hair removal agent, a moisturizing agent, an anti-wrinkle agent, an anaesthetic, a lip coloring agent and a combination thereof.

18. The electricity generating device of claim 16, wherein the at least one active composition is disposed on or in one of the group selected from the at least one delivery electrode, the lip area, the conductive carrier, a separator attached to at least one electrode, an active composition delivery chamber and a combination thereof.

19. The electricity generating device of claim 15, further comprising at least one power source.

20. The electricity generating device of claim 19, wherein the at least one power source is a flexible thin layer electrochemical cell.

21. The electricity generating device of claim 14, further comprising a heating means.

22. The electricity generating device of claim 14, wherein lip augmentation comprises at least one of enhancing the lips, resizing, plumping, reshaping, enlargement of the lips, improving symmetry, lifting of the lips, reducing lip area wrinkles and reducing wrinkles around the mouth.

23. A kit for lip augmentation comprising:
an electricity generating device for lip augmentation; and
an active composition for facilitating lip augmentation.
